(19) United States
(12) Patent Application Publication
(10) Pub. No.: US 2019/0317074 A1
(43) Pub. Date: Oct. 17, 2019

(54) APPARATUS AND METHOD FOR THE DETERMINATION OF SEPSIS RISK USING COMPONENTS OF EXHALED BREATH

(71) Applicant: Vail Scientific L.L.C., Inver Grove Heights, MN (US)

(72) Inventors: Carter R. Anderson, Inver Grove Heights, MN (US); Russell L. Morris, Lindstrom, MN (US); Thomas W. Burke, Plymouth, MN (US); Clayton J. Anderson, Lakeville, MN (US)

(21) Appl. No.: 16/381,960

(22) Filed: Apr. 11, 2019

Related U.S. Application Data
(60) Provisional application No. 62/761,941, filed on Apr. 13, 2018.

(51) Int. Cl. G01N 33/497; A61B 5/08; G01N 33/00; A61B 5/00

(52) U.S. Cl. CPC G01N 33/497; A61B 5/082; G16H 50/30; A61B 5/412; G01N 33/0037

(57) ABSTRACT

Disclosed herein are methods and devices for measurement of carbon dioxide and nitric oxide in exhaled breath of patients at risk for or suffering from sepsis. Carbon dioxide and nitric oxide levels may be measured in controlled rate breath and/or end tidal breath, and a risk score for the patient developing or having sepsis may be determined. In many embodiments, carbon dioxide and nitric oxide measurements are determined simultaneously in a single breath, and by a single measurement apparatus, while other embodiments measure nitric oxide and carbon dioxide levels using separate measure apparatuses and/or in different breaths of the patient.

### CROSS-REFERENCE TO RELATED APPLICATIONS

This application claims benefit of priority pursuant to 35 U.S.C. § 119(e) of U.S. provisional patent application No. 62/761,941 entitled “Apparatus and Method for the Noninvasive Determination of Sepsis Risk,” filed on 13 Apr. 2018, which is hereby incorporated by reference in its entirety.

### FIELD

The disclosed processes, methods, devices, and systems are directed to non-invasive, accurate, and reliable diagnosis of conditions associated with systemic bacterial infections, such as sepsis and risk of developing same.

### BACKGROUND

Every year, severe sepsis strikes more than a million Americans. It is estimated that between 28 and 50 percent of these people die, more than the number from prostate cancer, breast cancer and AIDS combined. The Agency for Healthcare Research and Quality lists sepsis as the most expensive condition treated in U.S. Hospitals, costing more than $20 billion in 2011.

Sepsis and septic shock is a serious medical condition caused by an overwhelming immune response to infection. Early diagnosis of sepsis has been shown to increase patient survival via appropriate treatment and decrease hospital stay/costs. Unfortunately, the currently available methods for diagnosing sepsis rely on detection of symptoms that only become evident after the infection has progressed to dangerous levels. It has been estimated that as many as 80% of lives lost to sepsis could be saved if a device for more rapid analysis was available.

Another problem associated with sepsis diagnosis results in potential for ‘over’ diagnosis, where patients are treated for sepsis despite not actually having it. Current methods for rapid diagnosis of sepsis (using the qSOFA method, for example) can result in patients being administered antibiotics when they do not actually need them. The qSOFA score relies on assessing three different criterion—mentation, respiratory rate, and blood pressure. While the qSOFA score may be rapidly calculated at the patient's bedside, it does not directly detect or assess the presence of microbes in the blood. A recent publication suggests that 56.7% of antibiotics administered to ER patients meeting qSOFA criteria are inappropriate.

There have been many attempts to characterize sepsis risk in a patient by assessment of various biomarkers in a blood sample. For example, US application 2018/0291449 proposes use of a panel of blood biomarkers for use to distinguish sepsis from non-infectious sources of inflammation. However, blood tests are ‘invasive’ tests that require a sample be drawn from a patient and sent to a lab, and typically the biomarker measurement requires a sophisticated bench-top analysis instrument.

There would be advantage in speed to assessment of sepsis if a point of care device could be devised. Such a method would eliminate the time involved in sending a sample to a hospital laboratory. Patent application 2018/0180589 describes a device and method where a blood sample could be collected and analyzed at a remote ‘point of care’ location. However, most proposed point of care methods still require blood sampling and sophisticated chemical analysis of the collected blood.

The overproduction of nitric oxide (NO) during sepsis is possibly the most important cause of the vasopressor-resistant hypotension which characterizes septic shock. Consequently, efforts have been undertaken to develop methods for analysis of NO and nitrosothiols in drawn blood samples as a means to detect the onset of sepsis in at-risk patients. A significant limitation to this approach rests in the instability of NO in the blood and resulting short half-life. Others have investigated use of sensors placed directly into the blood stream to detect NO. Using this technique in pre-clinical animal testing, the relationship between blood concentrations of NO and the onset of sepsis has been confirmed in a murine pneumonia model. However, this approach requires an invasive blood measurement that would be prone to significant limitations (instability, calibration drift, difficulty in placement, etc.).

There is obvious value in obtaining a non-invasive specimen for determining levels of biomarkers for disease states. Cutaneous and transcutaneous measurements for NO have been proposed for noninvasive sepsis diagnosis. In application 2018/002094, a non-invasive system and method to detect nitric oxide concentration in skin is described for detecting a sepsis condition. However, such a method may be unreliable owing to the fact nitric oxide can be generated in skin at varying levels in healthy, non-septic individuals as a result of simple environmental temperature change, thus confounding the analysis. Application WO 2017023500 proposes a transcutaneous measurement for NO and other blood gases for the noninvasive diagnosis of sepsis. However, as noted above, NO can be generated in skin for reasons related to temperature change rather than sepsis, and transcutaneous measurements of other blood gases are known to be temperature sensitive as well. Thus, both cutaneous and transcutaneous measurements are susceptible to error.

Another approach to noninvasive NO measurement is via gases measured in exhaled breath. In animal models, high concentrations of NO in exhaled breath have been successfully correlated to artificially induced sepsis caused by the administration of endotoxins. However, subsequent evaluations of humans with actual septic shock indicated that NO in exhaled breath only correlated with respiratory derived sepsis infections, and did NOT correlate with non-respiratory derived sepsis infections. (See, e.g., J ten Oever, et al., Pulmonary infection, and not systemic inflammation, accounts for increased concentrations of exhaled nitric oxide in patients with septic shock, 7 no. 3 J. Breath Res. (September 2013)). Consequently, “false negatives” would result from testing of septic patients with non-respiratory derived sepsis. The percentage of false negatives could be significant. For instance, in the Oever study, sixty percent of patients in the study had non-respiratory sepsis, which could not be detected by measuring exhaled NO concentration alone. A diagnostic technique that misses sixty percent of potential sepsis cases may be of limited value. Further, in this study, NO concentrations did not correlate with markers of sepsis disease severity, systemic inflammation, and haemodynamic instability. Additionally, high NO concentration in exhaled breath can also be impacted by non-sepsis medical conditions such as asthma or COPD, or patient habits such as smoking, increasing the rate of false positives. Thus, exhaled breath NO measurements alone are inadequate for the successful diagnosis of sepsis in humans, and no devices have been FDA approved for such an assessment.

Another non-invasive approach that has been investigated for the detection of sepsis, utilized the measurement of Carbon Dioxide (CO₂) in exhaled breath. In clinical investigations, low breath CO₂ levels were the strongest predictors of sepsis, severe sepsis, and mortality. There were also positive correlations between CO₂ in breath and serum bicarbonate, anion gap, and lactate. However, CO₂ levels in breath for example, end-tidal CO₂ or ETCO₂ can also be associated with non-sepsis related medical conditions including hyperventilation, hypoventilation, cardiac output, and others. While the clinical study successfully correlated low ETCO₂ levels with sepsis, these same low ETCO₂ levels could also be caused by many other conditions such as anxiety induced hyperventilation, thus giving “false positives”. Moreover, ETCO₂ measurements alone are unable to confirm the presence of an infection, a necessary component of sepsis. Thus, ETCO₂ measurements alone are inadequate for the successful diagnosis of sepsis. An important element to this disclosure includes an exhaled breath carbon dioxide measurement means enabling detection of sepsis with reduced numbers of “false positives”.

Applicants herein describe techniques and devices for a non-invasive measurement of nitric oxide levels in a patient. In contrast to existing devices and techniques, the described techniques and devices enable detection of sepsis that is both respiratory and non-respiratory derived (e.g. reducing false negatives for patients with non-respiratory sepsis), while concurrently reducing “false positives” associated with temperature change or with patients that may be asthmatic, have COPD, or have a smoking habit.

### SUMMARY

A sepsis assessment device is disclosed. The sepsis assessment device includes a fluid input in fluid communication with a respiratory tract of a patient, wherein the fluid input receives an exhaled breath from the patient; a NO measurement device that determines a NO concentration in the exhaled breath; a CO₂ measurement device that determines a CO₂ concentration in the exhaled breath; an input/output interface; a processing element in electrical communication with the CO₂ and NO measurement devices and the input/output interface, wherein the processing element receives a first signal corresponding to the NO concentration; receives a second signal corresponding to the CO₂ concentration; determines a sepsis risk score based on the first signal and the second signal; and transmits the sepsis risk score to the input/output interface.

A method for determining the risk of sepsis in a patient is disclosed. The method includes collecting an exhaled breath sample; regulating the flow rate of the exhaled breath sample; determining a CO₂ concentration in the exhaled breath sample; determining a NO concentration in the exhaled breath sample; correlating the NO concentration and the CO₂ concentration to a sepsis risk score; developing a patient treatment regime based on the sepsis risk score.

### BRIEF DESCRIPTION OF THE DRAWINGS

FIG. 1 is a schematic diagram of one embodiment of a sepsis diagnostic device.

FIG. 2 is a schematic diagram of one method of determining a sepsis risk score using the device of FIG. 1.

FIG. 3 is a graph of NO concentration versus CO₂ concentration showing Risk Score as a percentage of the likelihood of developing sepsis.

### DETAILED DESCRIPTION

Disclosed herein are improved, non-invasive methods and devices to aid in predicting the possibility of sepsis in a human patient at risk for having or developing sepsis. The disclosed devices and methods may also aid in predicting the risk of progression of sepsis to severe sepsis and/or septic shock in a human patient.

Applicants' methods and devices provide for the measurement of nitric oxide (NO) and carbon dioxide (CO₂) in exhaled breath to aid in assessing sepsis. Applicants have discovered that the combination of concentrations for both exhaled breath nitric oxide and exhaled breath carbon dioxide, and the unique relationship between them, provide a specificity for sepsis detection that is surprisingly enhanced (and unavailable) compared to other methods. In many embodiments, sepsis may be determined by analyzing the unique relationship between the concentrations of both nitric oxide and carbon dioxide in exhaled breath. Consequently, Applicants' disclosed methods and devices are unique in the ability to combine the confirmation of infection (via NO in exhaled breath), and metabolic disorder (via CO₂ in exhaled breath) that is necessary for the successful detection of sepsis risk in a patient. Further, the presently disclosed NO concentrations and CO₂ measurements correlate with markers of sepsis disease severity, systemic inflammation, and haemodynamic instability.

Disclosed herein are techniques to assess and control the way the exhaled breath NO sample is collected for sepsis assessment. In a preferred approach, the assessment of NO levels in exhaled breath is determined after the patient exhales in a controlled manner. For example, in most embodiments the patient preferably exhales at slower rate and, therefore, for a longer time than is required for other methods. Applicants have discovered that this slow, controlled rate allows for more accurate identification of the concentration of NO in the fraction of exhaled breath that has been in closest contact with lung tissue affected by sepsis.

The disclosed methods and devices analyze NO amounts when the patient breathes at less than about 200 ml/sec and/or for a time that is more than about 3 sec. In most cases, the volume of a breath of a human adult is about 500 ml. In many embodiments, the disclosed devices and methods may aid in controlling the rate of a patient's breath to enhance the accuracy of diagnosing sepsis in a patient. In most embodiments, the patient's breath may be exhaled at less than about 200 ml/sec, 190 ml/sec, 180 ml/sec, 170 ml/sec, 160 ml/sec, 150 ml/sec, 140 ml/sec, 130 ml/sec, 120 ml/sec, 110 ml/sec, 100 ml/sec, 90 ml/sec, 80 ml/sec, 70 ml/sec, 60 ml/sec, 50 ml/sec, 40 ml/sec, 30 ml/sec or 20 ml/sec, and greater than about 10 ml/sec, 20 ml/sec, 30 ml/sec, 40 ml/sec, 50 ml/sec, 60 ml/sec, 70 ml/sec, 80 ml/sec, 90 ml/sec, 100 ml/sec, 110 ml/sec, 120 ml/sec, 130 ml/sec, 140 ml/sec, 150 ml/sec, 160 ml/sec, 170 ml/sec, 180 ml/sec, and 190 ml/sec. When collected and measured in this manner, the device and method of this disclosure may analyze NO in the breath exhaled by a patient at a controlled rate of less than 200 ml/sec for a time period of at least 2 seconds. More preferably, it may collect NO concentration at a controlled exhale rate less than 100 ml/sec for a time period of at least 4 seconds. Most preferably, it may collect NO concentration at a controlled rate less than 50 ml/sec for a time period at least 6 seconds. For the purposes of this disclosure, a ‘controlled exhale’ constitutes a defined exhaled breath rate for a defined minimum period of time. An advantage of using a slow controlled exhale (e.g. less than 50 ml/sec) for sepsis diagnosis includes the opportunity to use untreated ambient air for inhale without the need to scrub the inhaled air to remove atmospheric NO.

An alternative approach for collecting NO concentration in exhaled breath in this disclosure is to use end-tidal NO (ETNO) concentration when the patient exhales in a normal breath pattern. For the purposes of this disclosure, end tidal NO (ETNO) or end-tidal CO₂ (ETCO₂) is defined as the maximum concentration of NO or CO₂ generally found at the end of the normal exhaled breath cycle.

The device of this disclosure may measure NO and CO₂ in exhaled breath using electrochemical or spectroscopic sensing means. Such sensing methods are well known to those skilled in the art, and currently utilized in separate devices for different clinical purposes. An example of an NO detector or measurement device is the NIOX® VERO supplied by Circassia Pharmaceuticals, Inc. An example of a CO₂ detector or measurement device is the CAPNOCHECK® II supplied by Smiths Medical. In most embodiments of this disclosure, the disclosed devices and methods may measure both NO and CO₂ in an exhaled breath. In some embodiments, the NO and CO₂ concentrations may be measured in separate breaths. In some embodiments, separate devices may be used to measure NO and CO₂ in the same or different breaths. For example, disclosed herein is the use of two separate devices for measuring NO and CO₂ respectively. In some embodiments, a first measurement on a first device is followed by a second measurement on a second device. Whether using a single instrument or two, both the NO and CO₂ values are used for the assessment of sepsis risk.

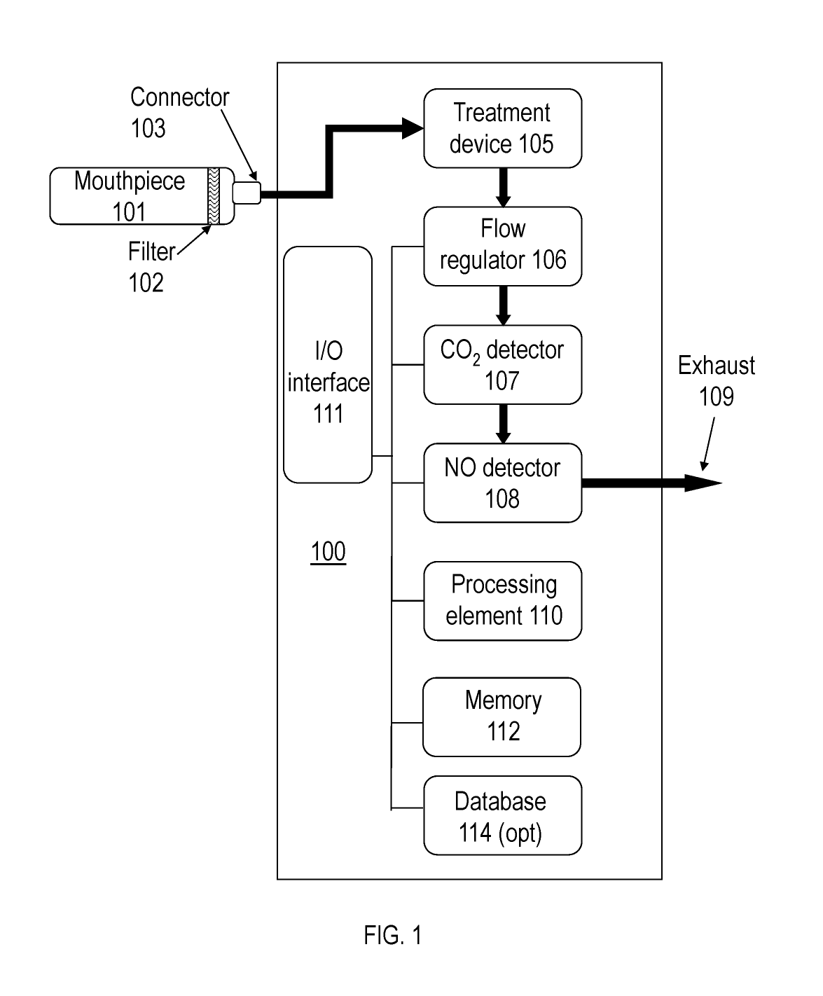

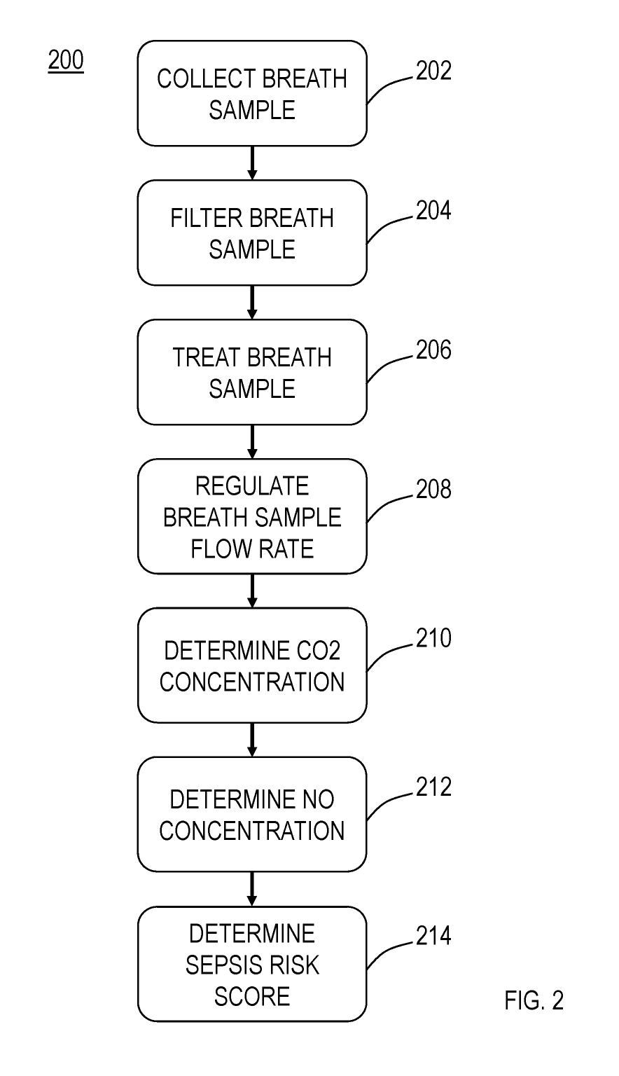

FIG. 1 illustrates an embodiment of a sepsis assessment device 100 to assess sepsis risk by determining both exhaled CO₂ and NO concentrations. FIG. 2 illustrates a method 200 of assessing a patient's sepsis risk using exhaled CO₂ and NO concentrations. The method 200 may be executed on the sepsis assessment device 100. The method 200 may also be implemented independent of the sepsis assessment device 100. For example, the method 200 may be executed with discrete devices that measure NO and CO₂, and the respective concentrations may be inputted to a separate processing element to determine a risk score. Likewise, the sepsis assessment device 100 may execute methods different from or in addition to the method 200. The method 200 and the sepsis assessment device 100 are presented together for the sake of brevity but do not rely on one another.

Some or all of the components of the device 100 may be housed in a single integrated unit, or some or all of the components may be separate components in fluid and/or electrical communication with one another as described. The order in which the components of the sepsis assessment device 100 receive exhaled breath may vary from the order presented without departing from the present disclosure. The presented order is exemplary only, in order to facilitate understanding of the sepsis assessment device 100 and the methods of using it. Likewise the presented order of the operations of method 200 is exemplary only, and not limiting. Other orders of operation are contemplated and may be used without departing from the present disclosure.

The one or more device processing elements 110 are substantially any type of electronic device capable of processing, receiving, and/or transmitting instructions. For example, the processing element 110 may be a microprocessor or a microcontroller, a central processing unit (CPU) a graphics processing unit (GPU), a field programmable gate array (FPGA). Additionally or alternatively, select data processing steps or processes may be performed by one processing element 110 with other data processing steps performed by different processing elements 110, where the different processing elements 110 may or may not be in communication with each other.

The one or more memory components 112 may store electronic data used by the sepsis assessment device 100 to store instructions for the processing element 110, as well as to store data collected by the CO₂ and or NO detectors 107, 108, respectively. In some examples, the one or more memory components 112 may be one or more magnetic hard disk drives, solid state drives, magneto optical memory, flash memory, electrically erasable programmable read-only memory (“EEPROM”), erasable programmable read-only memory (“EPROM”), ferromagnetic RAM, holographic memory, printed ferromagnetic memory, or non-volatile memory. In other examples, memory 112 may be any volatile computer readable media device that requires power to maintain its memory state. In one example, memory 112 is random access memory (“RAM”). Other examples may include dynamic RAM, and static RAM, or a combination of one or more types of memory components.

Optionally, the sepsis assessment device 100 may include a database 114. The database may be any type of collection of records of data relating exhaled NO and CO₂ concentrations to sepsis risk. In one embodiment, the sepsis assessment device 100 may be in communication with an external database 114, such as a database stored on a remote server. The sepsis assessment device 100 may communicate with the database 114 on the remote server through the I/O interface 111. In one embodiment, the database 114 includes of information for previous patients having similar nitric oxide and carbon dioxide levels.

The I/O interface 111 provides communication to and from the sepsis assessment device 100 to exterior devices and/or users such as medical professionals. In one embodiment, the I/O interface is a display, such as a light emitting diode display, a liquid crystal display, cathode ray tube, plasma panel, ticker tape or other device that outputs data in a visual format. The I/O interface may include an annunciator, speaker or other auditory output. The I/O interface 111 may include one or more input buttons or sensors, a communication interface (such as Wi-Fi®, Ethernet, Bluetooth®, NFC, RFID, cellular, infrared or other optical communications, or the like), communication components (such as universal serial bus (USB) ports/cables), or the like. In various examples, the I/O interface 111 transmits sepsis risk scores, detector 107, 108 data such as CO₂ or NO concentrations, or other data. The I/O interface may receive data, such as biometric or demographic information about the patient (e.g., height, weight, age, blood pressure, pulse oxidation, pulse rate, race or ethnicity, socioeconomic status, or other information) that may be used in conjunction with CO₂ and/or NO data to determine a sepsis risk score. The I/O interface may communicate data to, or receive data from a remote computing device, such as a remote server. The I/O interface may also transmit data to a healthcare provider device such as a tablet, laptop, or desktop computer, or a user mobile device such as a smart phone. The sepsis assessment device 100 may record biometric or demographic information as well as information about exhaled NO and CO₂ concentrations, and sepsis diagnosis, severity and outcomes about patients to the database 114. The sepsis assessment device 100 may access such information to continually improve the accuracy of sepsis risk assessments.

The sepsis assessment device 100 may include a mouthpiece 101 adapted to collect the exhaled breath. The mouthpiece 101 may contain a filter 102. The mouthpiece 101 may be in fluid communication with a connector 103 that is also in fluid communication with a fluid input of the sepsis assessment device 100. The sepsis assessment device 100 may contain a treatment device 105 that receives the exhaled breath from the connector 103 and treats the exhaled breath to prepare it for further analysis. The treatment device 105 may pass the treated exhaled breath to a flow regulator 106. The flow regulator 106 may control the flow rate of the treated exhaled breath to other components of the sepsis assessment device 100 such as a CO₂ detector or measurement device 107 and/or an NO detector or measurement device 108. The sepsis assessment device 100 may then exhaust the treated exhaled breath. One or more processing elements 110 and one or more memory components may be in electrical communication with any of the flow regulator 106, the detectors 107, 108 and an input/output (I/O) interface. The processing element may read CO₂ and NO reading from the respective detectors 107, 108 and determine a sepsis risk score. The processing element 290 may output the sepsis risk score to the I/O interface.

The method 200 may begin with operation 202, where an exhaled breath sample is collected from a patient. The operation 202 may be executed by the sepsis assessment device 100. In one embodiment, the sepsis assessment device 100 may have a mouthpiece 101 that may be in fluid communication with the respiratory tract of a patient. In another embodiment, the mouthpiece 101 may be in fluid communication with a mechanical ventilator that is breathing for a patient. The mouthpiece 101 receives exhaled breath from the patient, either directly from the respiratory tract, or via a mechanical ventilator. The mouthpiece 101 is adapted to allow the passage of exhaled breath either from a patient's respiratory tract or a mechanical ventilator to the device 100.

The method 200 may proceed to operation 204 where the exhaled breath is filtered. Operation 204 may be executed by the filter 102. The mouthpiece 101 may have a filter 102 housed within it. The filter 102 may be a separate device in fluid communication with the mouthpiece 101. The filter 102 may be permanently housed within the mouthpiece 101, or it may be removable. The filter 102 may be adapted to remove particulate matter, moisture droplets, or the like from the exhaled breath before passing it to the connector 103.

The connector 103 may be in fluid communication with the mouthpiece 101 and/or the filter 102, and the device 100. The connector may be any type of suitable conduit that facilitates passage of exhaled breath from the mouthpiece 101 and/or filter 102 to the device 100. In various examples, the connector may be a tube, hose, pipe, duct, straw or other suitable structure adapted to pass exhaled breath from the mouthpiece 101 and/or the filter 102, to the device 100. In many embodiments, the mouthpiece 101, filter 102, and/or connector 103 may be replaceable and/or adaptable, such that the device 101 may be used by two or more different patients, or the same patient multiple times.

The method 200 may proceed to operation 206 where the exhaled breath is treated. A treatment device 105 may be used to execute operation 206. The treatment device 105 may receive exhaled breath from the connector 103. The treatment device 105 prepares the exhaled breath for further analysis. In one example, the treatment device 105 removes moisture from the exhaled breath. In various examples, the treatment device 105 may be a desiccant, a coalescing filter, a mechanical separator such as a cyclonic separator or screen, or any of these. The treatment device 105 may receive exhaled breath and output treated exhaled breath. The treatment device 105 may pass the treated exhaled breath to a flow regulator 106.

The method 200 may proceed to operation 208 where the flow rate of the treated exhaled breath is regulated. A flow regulator 106 may be used to execute operation 208. The flow regulator 106 may be any suitable device that can control the flow rate (either volumetric or mass flow), and/or pressure of the treated exhaled breath to downstream components of the sepsis assessment device 100. In various examples, the flow regulator may be a venturi, converging/diverging nozzle, an orifice, backpressure regulator, a forward pressure regulator, a mass flow controller, one or more valves and or actuators or biasing elements.

The method 200 may proceed to operation 210 where the CO₂ concentration or partial pressure in the treated exhaled breath is determined. The operation 210 may be executed by a CO₂ detector 107. The sepsis assessment device 100 may contain or be in fluid communication with a CO₂ detector. The CO₂ detector 107 may be any suitable device that can detect a concentration or partial pressure of CO₂ in the treated exhaled breath, and communicate a corresponding reading to the processing element 110 and/or the I/O interface 111. In one example, the CO₂ detector 107 is a capnography sensor that detects CO₂ concentration by measuring an amount of light (e.g., infrared light) absorbed from a light beam passing through the treated exhaled breath and correlates that absorption to a concentration or partial pressure of CO₂ in the treated exhaled breath. In various other examples, the CO₂ detector 108 uses chemiluminescence or electrochemical methods to detect CO₂ concentrations. The CO₂ detector 107 may be in electrical communication with an NO detector 108, for instance to adjust the CO₂ reading for the presence of NO in the treated exhaled breath.

The method 200 may proceed to operation 212 where the NO concentration in the treated exhaled breath is determined. The NO detector 108 may execute the operation 212. The sepsis assessment device 100 may contain or be in fluid communication with an NO detector 108. The CO₂ detector 107 and the NO detector 108 may be one device that can measure the concentration or partial pressure of both gases. The NO detector 108 may be any suitable device that can detect a concentration or partial pressure of NO in the treated exhaled breath, and communicate a corresponding reading to the processing element 110 and/or the I/O interface 111. In one example, the NO sensor is a sensor that detects NO concentration by measuring an amount of light (e.g., infrared light) absorbed from a light beam passing through the treated exhaled breath and correlates that absorption to a concentration or partial pressure of NO in the treated exhaled breath. In various other examples, the NO detector 108 uses chemiluminescence or electrochemical methods to detect NO concentrations. The NO detector 108 may be in electrical communication with the CO₂ sensor 107, for instance to adjust the CO₂ reading for the presence of NO in the treated exhaled breath.

The CO₂ detector 107 and the NO detector 108 may be arranged to receive the treated exhaled breath in any order. For example, the CO₂ detector 107 may receive the treated exhaled breath first, and then the NO detector 108 may receive the treated exhaled breath. In one example, the NO detector 108 may receive the treated exhaled breath followed by the CO₂ detector 107. In another example, the treated, exhaled breath may be passed in parallel to both detectors 107, 108, in any proportion. For instance, the treated exhaled breath may be split evenly between the detectors 107, 108. In another example, the treated exhaled breath may be split 51-99% to one detector 107, 108 and 1-49% to the other detector 107, 108, for example 55/45, 60/40, 65/35, 70/30 etc. There may be more than one flow regulator 106, each adapted to split and control the flow of the treated exhaled breath to one of the detectors 107, 108. After the treated exhaled breath is analyzed by both the CO₂ detector 107 and the NO detector 108, the treated exhaled breath may be exhausted from the sepsis assessment device 100.

Either the CO₂ detector and/or the NO detector may output signals that correspond to the respective concentrations of CO₂ and/or NO as controlled rate and/or end-tidal values. For example, the NO detector may output a signal corresponding to a controlled rate NO value and/or an end-tidal NO value. Likewise, the CO₂ detector may output a signal corresponding to a controlled rate CO₂ and end-tidal CO₂.

The method 200 may conclude with operation 214 where the sepsis risk score is determined and outputted. The one or more processing elements 112 may execute operation 214. The one or more processing elements 112 may be in electrical communication with the detectors 107, 108. The processing element 112 may receive electrical signals that correlate to a concentration or partial pressure of CO₂ and NO, respectively. The processing element 112 may determine a sepsis risk score from the CO₂ and NO concentration data.

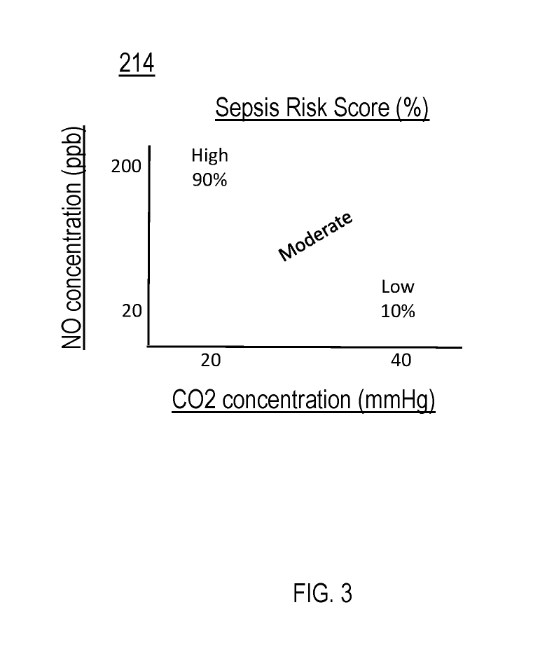

FIG. 3 illustrates one embodiment of operation 214 for determining a sepsis risk score. In the embodiment, a CO₂ concentration of 20 mmHg and an NO concentration of 200 ppb may indicate a 90% likelihood that a patient has sepsis. In the embodiment, a CO₂ concentration of 40 mmHg and an NO concentration of 20 ppb may indicate a 10% likelihood that a patient has sepsis. Other NO concentrations and CO₂ concentrations may fall between the risk values shown in FIG. 3. By determining both the CO₂ and the NO concentrations in exhaled breath, a sepsis risk score may be assigned to a patient.

The disclosed methods and devices may be used to assess a patient that is conscious or unconscious. In most embodiments, the patient is conscious when NO and CO₂ values are determined. In most embodiments, a patient may be assessed at any stage of sepsis, for example from early stage sepsis, for example where the patient may present with a fever and/or increased heart rate, to septic shock. In most cases, a patient in an early sepsis stage will not require mechanical ventilation. Assessment at this stage may also allow for early treatment of a sepsis diagnosis with antibiotic treatment. In some cases, when a patient is in early stage of sepsis, the patient is conscious and may be better able to exhale in a controlled pattern that is different from a normal breath exhale. Those skilled in the art will recognize the utility of the disclosed methods and devices for use with patients of more advanced stage sepsis, for example those patients requiring mechanical ventilation.

One feature of this disclosure relates to how individual test results can be correlated to a sepsis diagnosis. Information from the sepsis assessment device 100 is intended to alert clinicians as to whether there is a need to quickly treat the patient for sepsis. In most cases, a diagnosis of sepsis from the disclosed devices and methods may likely result in treating the patient with one or more antibiotics. Information from this disclosure may also inform clinicians as to the risk this patient may have for developing severe sepsis or going into septic shock. Thus, results such as a sepsis risk score resulting from the disclosed methods and devices, may be used to develop treatment plans or regimes for the patient. In various examples, if the sepsis risk score is high, the score may be used to develop a more aggressive treatment regime, for example one or more of transfer to an intensive care unit, intravenous delivery of antibiotics, intravenous delivery of fluids, high dose antibiotics, broad spectrum antibiotics, oxygen therapy, etc. The described methods may indicate a patient has or is at risk of sepsis based on analyzing and measuring an exhaled breath NO measurement and an exhaled breath CO₂ measurement. In many embodiments, a positive sepsis diagnosis may occur wherein a NO concentration is greater than about 40 ppb concentration when measured as an average, median or maximum concentration in a controlled rate exhalation and a CO₂ reading is less than about 25 mmHg partial pressure concentration when measured as an end tidal value (ETCO₂). In many embodiments a patient may be determined to have sepsis wherein the patient has an average NO reading of greater than about 30 ppb, 31 ppb, 32 ppb, 33 ppb, 34 ppb, 35 ppb, 36 ppb, 37 ppb, 38 ppb, 39 ppb, 40 ppb, 41 ppb, 42 ppb, 43 ppb, 44 ppb, 45 ppb, 46 ppb, 47 ppb, 48 ppb, 49 ppb, 50 ppb, 51 ppb, 52 ppb, 53 ppb, 54 ppb, 55 ppb, 60 ppb, 65 ppb, 70 ppb, 75 ppb, 80 ppb, 85 ppb, 90 ppb, 95 ppb, 100 ppb, 105 ppb, 110 ppb, 115 ppb, 120 ppb, 125 ppb, 130 ppb, 135 ppb, 140 ppb, 155 ppb, 160 ppb, 165 ppb, 170 ppb, 175 ppb, 180 ppb, 185 ppb, 190 ppb, 195 ppb, 200 ppb, 210 ppb, 215 ppb, 220 ppb, 225 ppb, 230 ppb, 235 ppb, 240 ppb, and 245 ppb and less than about 300 ppb, 290 ppb, 280 ppb, 270 ppb, 260 ppb, 250 ppb, 240 ppb, 230 ppb, 220 ppb, 210 ppb, 200 ppb, 190 ppb, 180 ppb, 170 ppb, 160 ppb, 150 ppb, 140 ppb, 130 ppb, 120 ppb, 110 ppb, 100 ppb, 90 ppb, 80 ppb, 70 ppb, 60 ppb, 55 ppb, 50 ppb, 49 ppb, 48 ppb, 47 ppb, 46 ppb, 45 ppb, 44 ppb, 43 ppb, 42 ppb, 41 ppb, 40 ppb, 39 ppb, 38 ppb, 37 ppb, 36 ppb, 35 ppb, 34 ppb, 33 ppb, 32 ppb, or 31 ppb, and a CO₂ reading of less than about 50 mmHg, 49 mmHg, 48 mmHg, 47 mmHg, 46 mmHg, 45 mmHg, 44 mmHg, 43 mmHg, 42 mmHg, 41 mmHg, 40 mmHg, 39 mmHg, 38 mmHg, 37 mmHg, 36 mmHg, 335 mmHg, 34 mmHg, 33 mmHg, 32 mmHg, 31 mmHg, 30 mmHg, 29 mmHg, 28 mmHg, 27 mmHg, 26 mmHg, 25 mmHg, 24 mmHg, 23 mmHg, 22 mmHg, 21 mmHg, 20 mmHg, 19 mmHg, 18 mmHg, 17 mmHg, 16 mmHg, or 15 mmHg, and greater than about 10 mmHg, 14 mmHg, 15 mmHg, 16 mmHg, 17 mmHg, 18 mmHg, 19 mmHg, 20 mmHg, 21 mmHg, 22 mmHg, 23 mmHg, 24 mmHg, 25 mmHg, 26 mmHg, 27 mmHg, 28 mmHg, 29 mmHg, 30 mmHg, 31 mmHg, 32 mmHg, 33 mmHg, 34 mmHg, 35 mmHg, 36 mmHg, 37 mmHg, 38 mmHg, 39 mmHg, 40 mmHg, 41 mmHg, 42 mmHg, 43 mmHg, 44 mmHg, 45 mmHg, 46 mmHg, 47 mmHg, 48 mmHg, 49 mmHg, and 50 mmHg. The risk of developing sepsis, or of having sepsis may increase as exhaled NO concentrations rise above 40 ppb and the CO₂ concentrations decrease from 40 mmHg.

Assessing values for NO concentration and CO₂ concentration in exhaled breath of a patient, may aid in providing a risk score for the patient developing sepsis in the future if the patient is not treated for a bacterial infection. In many embodiments, the risk score may be represented by a percentage value, for example a value between 0 and 100. The percentage value may correspond to a probability that the patient has, or may develop, sepsis. In some embodiments, a patient whose NO and CO₂ concentrations in exhaled breath indicate a low risk of imminent sepsis, the risk score value may be less than about 10. Those skilled in the art would recognize a low risk score would allow time for additional more confirmatory testing for infection or to defer antibiotic or blood filtration treatment.

It is also contemplated that the devices and methods of this disclosure incorporates an improved method for interpreting results. Conventionally, clinical instruments produce numerical concentration results and require a clinician to compare those results to a normal range. Because this disclosure requires interpretation of more than one result, confusion may occur if one parameter falls within a normal range while the other falls outside a normal range. An embodiment of this disclosure is one where the device uses an algorithm to output a percent risk that a patient has sepsis based on previous clinical trial results for that segment of patients having similar NO and CO₂ readings.

The disclosed devices and methods aid in diagnosing sepsis in a patient that may have sepsis or be at risk for developing sepsis. For example, results from this method may predict the likelihood that a given patient may have microbiologically confirmed sepsis and/or determine the risk that a given patient may be on a course that may lead to septic shock.

The disclosed methods and devices may be used to assess a patient who is conscious or unconscious. In most embodiments, the patient is conscious when NO and CO₂ values are determined. In most embodiments, a patient may be assessed at any stage of sepsis, for example from early stage sepsis, for example where the patient may present with a fever and/or increased heart rate, to septic shock. In most cases, a patient in an early sepsis stage may not require mechanical ventilation. Assessment at this stage may also allow for early treatment of a sepsis diagnosis with antibiotic treatment. In some cases, when a patient is in early stage of sepsis, the patient is conscious and may be better able to exhale in a controlled pattern that is different from a normal breath exhale. Those skilled in the art may recognize the utility of the disclosed methods and devices for use with patients of more advanced stage sepsis, for example those patients requiring mechanical ventilation.

While all embodiments of this disclosure include the non-invasive exhaled breath measurement of CO₂ and NO, it is contemplated that other non-invasive measurements could be incorporated as well. For example, a pulse oximetry result, a respiratory rate, a blood pressure reading, a patient temperature reading, or a cognitive assessment reading could be added to help ascertain the patient's risk for sepsis. Of these, preferred additional measurements include diastolic blood pressure, systolic blood pressure, and oxygen saturation %. Such measurements could be incorporated into the device of this disclosure, or otherwise collected by other devices and results input into the device of this disclosure for enhanced sepsis risk assessment.

While multiple embodiments are disclosed, still other embodiments of the present disclosure may become apparent to those skilled in the art from the following detailed description. As may be apparent, the disclosure is capable of modifications in various obvious aspects, all without departing from the spirit and scope of the present disclosure. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, may control.

Although the present disclosure has been described with a certain degree of particularity, it is understood the disclosure has been made by way of example, and changes in detail or structure may be made without departing from the spirit of the disclosure as defined in the appended claims.

1. A sepsis assessment device comprising:
a fluid input in fluid communication with a respiratory tract of a patient, wherein the fluid input receives an exhaled breath from the patient;
a NO measurement device that determines a NO concentration in the exhaled breath;
a CO₂ measurement device that determines a CO₂ concentration in the exhaled breath;
an input/output interface;
a processing element in electrical communication with the CO₂ and NO measurement devices and the input/output interface, wherein the processing element receives a first signal corresponding to the NO concentration;
receives a second signal corresponding to the CO₂ concentration;
determines a sepsis risk score based on the first signal and the second signal; and
transmits the sepsis risk score to the input/output interface.

2. The device of claim 1, wherein the NO measurement device measures one of controlled rate NO and end-tidal NO.

3. The device of claim 1, wherein the CO₂ measurement device measures one of controlled rate CO₂ and end-tidal CO₂.

4. The device of claim 1, wherein the NO measurement device and the CO₂ measurement device are the same device that can measure both NO and CO₂.

5. The device of claim 1, wherein the determination of the sepsis risk score includes comparing the NO concentration and the CO₂ concentration against a database of information for previous patients having similar nitric oxide and carbon dioxide levels.

6. The device of claim 5, wherein the processing element uses information from the database to improve accuracy of the sepsis risk score.

7. A method for determining the risk of sepsis in a patient comprising:
collecting an exhaled breath sample;
regulating the flow rate of the exhaled breath sample;
determining a CO₂ concentration in the exhaled breath sample;
determining a NO concentration in the exhaled breath sample;
correlating the NO concentration and the CO₂ concentration to a sepsis risk score;
developing a treatment regime for the patient that is based on the sepsis risk score.

8. The method of claim 7, wherein the CO₂ concentration is obtained by a controlled rate measurement and the NO concentration is obtained by a controlled rate measurement.

9. The method of claim 7, wherein the CO₂ concentration is obtained by an end tidal measurement and the NO concentration is obtained by an end tidal measurement.

10. The method of claim 7, wherein the CO₂ concentration is obtained by a controlled rate measurement and the NO concentration is obtained by an end tidal measurement.

11. The method of claim 7, wherein the CO₂ concentration is obtained by an end tidal measurement and the NO concentration is obtained by a controlled rate measurement.

12. The method of claim 7, wherein the sepsis risk score is used to diagnose sepsis in the patient.

13. The method of claim 7, wherein exhaled breath concentrations of CO₂ below 25 mmHg and exhaled breath concentrations of NO above 25 ppb are indicative of sepsis risk greater than about 10%.

14. The method of claim 7, wherein exhaled breath concentrations of CO₂ below 25 mmHg, and exhaled breath concentrations of NO above 40 ppb are indicative of sepsis risk greater than about 10%.

15. The method of claim 7, wherein the sepsis risk score is based on a comparison of NO and CO₂ readings against a database of NO and CO₂ readings and scores for previous patients having similar NO and CO₂ levels.

16. The method of claim 15, wherein the previous patient is the same patient, and the readings include a trend over time.

17. The method of claim 15, wherein a processing element uses information from the database to improve accuracy of the sepsis risk score.

18. The method of claim 15, wherein the database is contained within a sepsis assessment device.

19. The method of claim 15, wherein the database is remotely accessed from a sepsis assessment device.
